(19) United States
(12) Patent Application Publication
(10) Pub. No.: US 2025/0250228 A1
(43) Pub. Date: Aug. 7, 2025

(54) METHODS FOR THE PREPARATION OF 5-CHLORO-2-((ETHOXYCARBONYL)AMINO)-3-METHYLBENZOIC ACID

(71) Applicants: FMC CORPORATION, Philadelphia, PA (US); FMC IP Technology GmbH, Neuhausen (CH)

(72) Inventors: Yuanjun Di, Shanghai (CN); Shuren Ji, Shanghai (CN); Jie Luan, Shanghai (CN); Jianhua Mao, Shanghai (CN); Wangrong Shi, Shanghai (CN); Hao Wang, Shanghai (CN)

(21) Appl. No.: 18/856,268

(22) PCT Filed: Apr. 13, 2023
(86) PCT No.: PCT/US2023/018415

§ 371 Date: Oct. 11, 2024

Related U.S. Application Data
(60) Provisional application No. 63/330,813, filed on Apr. 14, 2022.

(51) Int. Cl. C07C 271/28; C07C 269/06

(52) U.S. Cl. CPC C07C 271/28; C07C 269/06

(57) ABSTRACT

Described herein are methods of synthesizing 5-chloro-2-((ethoxycarbonyl)amino)-3-methylbenzoic acid and derivatives thereof.

### CROSS-REFERENCE TO RELATED APPLICATIONS

This application claims the benefit of U.S. Provisional Application No. 63/330,813 filed Apr. 14, 2022.

### FIELD OF INVENTION

This disclosure is directed to methods of synthesizing 5-chloro-2-((ethoxycarbonyl)amino)-3-methylbenzoic acid and derivatives thereof. Compounds prepared by the methods disclosed herein are particularly useful for preparation of certain anthranilamide compounds that are of interest as insecticides, such as, for example, the insecticides chlorantraniliprole and cyantraniliprole.

### BACKGROUND

The present disclosure provides novel methods useful for preparing 5-chloro-2-((ethoxycarbonyl)amino)-3-methylbenzoic acid and derivatives thereof. Methods to prepare 5-chloro-2-((ethoxycarbonyl)amino)-3-methylbenzoic acid are known in the art, such as those described in U.S. Pat. No. 8,153,844. However, these known methods are costly and wasteful.

In such a known method, chlorine is produced on site by 30% hydrogen peroxide and 37% hydrochloric acid. The reaction rate is controlled by the dose rate of hydrogen peroxide. Typically, the reaction is conducted in acetic acid solution at 30-35° C. for 3-5 hours. After reaction, the product is given by plentiful water dilution of the reaction mixture, thereby producing much wastewater. Acetic acid is left in the wastewater and is difficult to recover.

Compared to conventional processes, the benefits of the methods of the present disclosure are numerous and include improved reduced waste, recovery and/or recycling of solvent, and reduced cost.

### BRIEF DESCRIPTION

In one aspect, provided herein is a method of preparing a compound of Formula (II)

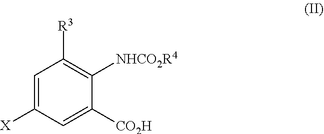

wherein R³ is CH₃ or Cl;
R⁴ is C₁-C₆ alkyl or C₃-C₆ alkenyl, each optionally substituted with up to 3 halogen and up to 1 phenyl; and
X is Cl or Br, the method comprising:
   (I) forming a mixture comprising:
      A) a compound of Formula (I)

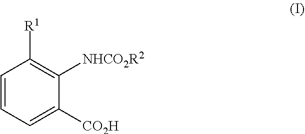

wherein R¹ is CH₃ or Cl; and
R² is C₁-C₆ alkyl or C₃-C₆ alkenyl, each optionally substituted with up to 3 halogen and up to 1 phenyl; and
   B) an aqueous solvent, wherein the aqueous solvent is present in a concentration less than or equal to about 90% v/v;
(II) introducing a halogenation agent to the mixture;
(III) optionally removing the halogenation agent from the mixture; and
(IV) optionally cooling the mixture to a temperature in a range of from about 0° C. to about 5° C.

### DETAILED DESCRIPTION OF THE DISCLOSURE

Described herein are methods of synthesizing 5-chloro-2-((ethoxycarbonyl)amino)-3-methylbenzoic acid and derivatives thereof.

The embodiments of this disclosure include:

Embodiment 1. A method of preparing a compound of Formula (II)

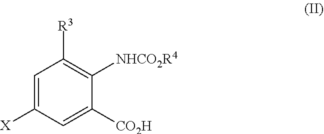

wherein R³ is CH₃ or Cl;
R⁴ is C₁-C₆ alkyl or C₃-C₆ alkenyl, each optionally substituted with up to 3 halogen and up to 1 phenyl; and
X is Cl or Br, the method comprising:
   (I) forming a mixture comprising:
      A) a compound of Formula (I)

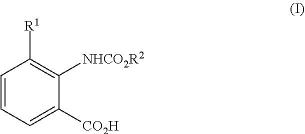

wherein R¹ is CH₃ or Cl; and
R² is C₁-C₆ alkyl or C₃-C₆ alkenyl, each optionally substituted with up to 3 halogen and up to 1 phenyl; and
   B) an aqueous solvent, wherein the aqueous solvent is present in a concentration less than or equal to about 90% v/v;
(II) introducing a halogenation agent to the mixture;
(III) optionally removing the halogenation agent from the mixture; and
(IV) optionally cooling the mixture to a temperature in a range of from about 0° C. to about 5° C.

Embodiment 2. The method of embodiment 1, wherein R³ and X are each Cl.

Embodiment 3. The method of any of embodiments 1-2, wherein R³ is CH₃ and X is Cl.

Embodiment 4. The method of any of embodiments 1-3, wherein R⁴ is C₁-C₂ alkyl.

Embodiment 5. The method of any of embodiments 1-4, wherein R¹ is CH₃.

Embodiment 6. The method of any of embodiments 1-5, wherein R² is C₁-C₂ alkyl.

Embodiment 7. The method of any of embodiments 1-6, wherein R³ is CH₃, X is Cl, and R⁴ is ethyl.

Embodiment 8. The method of any of embodiments 1-7, wherein the aqueous solvent is present in a concentration less than or equal to about 85% v/v.

Embodiment 9. The method of any of embodiments 1-8, wherein the aqueous solvent is present in a concentration less than or equal to about 80% v/v.

Embodiment 10. The method of any of embodiments 1-9, wherein the aqueous solvent is an aqueous solution of acetic acid.

Embodiment 11. The method of any of embodiments 1-10, wherein the aqueous solvent does not comprise hydrogen peroxide.

Embodiment 12. The method of any of embodiments 1-11, wherein the aqueous solvent comprises a recovered and/or recycled aqueous solvent.

Embodiment 13. The method of any of embodiments 1-12, wherein the halogenation agent is a chlorination agent or a bromination agent.

Embodiment 14. The method of any of embodiments 1-13, wherein the halogenation agent is selected from the group consisting of Cl₂, Br₂, and combinations thereof.

Embodiment 15. The method of any of embodiments 1-14, wherein the method step of introducing a halogenation agent to the mixture comprises introducing the halogenation agent to the mixture at a temperature in a range of from about 0° C. to about 35° C.

Embodiment 16. The method of any of embodiments 1-15, wherein the method step of removing the halogenation agent from the mixture comprises bubbling N2 through the mixture.

Embodiment 17. The method of any of embodiments 1-16, wherein the method produces a solid product, and wherein the method further comprises a method step of filtering and/or washing the solid product.

Embodiment 18. The method of any of embodiments 1-18, wherein the method further comprises a method step of recovering and/or recycling the aqueous solvent.

Embodiment 19. The method of embodiment 19, wherein the method step of recovering and/or recycling the aqueous solvent recovers and/or recycles at least 50% v/v of the aqueous solvent.

Embodiment 20. The method of embodiment 18, wherein the method step of recovering and/or recycling the aqueous solvent recovers and/or recycles at least 75% v/v of the aqueous solvent.

Embodiment 21. The method of any of embodiments 1-20, wherein the method further comprises a method of step of recovering and/or recycling a mother liquid.

Embodiment 22. The method of embodiment 21, wherein the method step of recovering and/or recycling the mother liquid recovers and/or recycles at least 50% v/v of the mother liquid.

Embodiment 23. The method of embodiment 21, wherein the method step of recovering and/or recycling the mother liquid recovers and/or recycles at least 80% v/v of the mother liquid.

In one aspect, 5-chloro-2-((ethoxycarbonyl)amino)-3-methylbenzoic acid is prepared according to a method represented by Scheme 1.

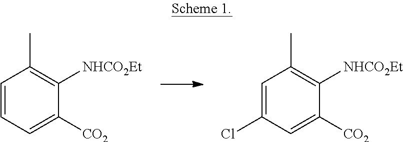

In one aspect, a compound of Formula II is prepared according to a method represented by Scheme 2. The R groups and X are as defined anywhere in this disclosure.

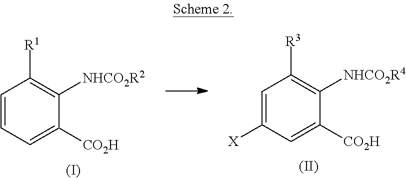

This aspect includes forming a mixture comprising a compound of Formula I and an aqueous solvent, introducing a halogenation agent to the mixture, optionally removing the halogenation agent from the mixture, and optionally cooling the mixture to a temperature in a range of from about 0° C. to about 5° C.

In some embodiments, the reaction of the mixture is complete after completion of a reaction time.

In some embodiments, the aqueous solvent is an aqueous solution of acetic acid. In some embodiments, the aqueous solvent does not comprise hydrogen peroxide. In some embodiments, the aqueous solvent comprises a recovered and/or recycled aqueous solvent.

Generally, any suitable amount of acetic acid may be used in the solvent. In many embodiments, the amount and/or concentration of acetic acid is relatively low to reduce usage of acetic acid and waste.

In some embodiments, the aqueous solvent is a mother liquid. In some embodiments, the aqueous solvent is a recovered and/or recycled mother liquid. In these embodiments, the mother liquid is a mixture of the aqueous solvent, optional residual products from earlier reactions, and optional impurities from earlier reactions. In many embodiments, recovering a mother liquid from an earlier reaction and recycling it as an aqueous solvent in a subsequent reaction allows for reduced waste and increased yield.

In some embodiments, the aqueous solvent is present in a concentration less than or equal to about 90% v/v, about 85% v/v, or about 80% v/v. In some embodiments, the aqueous solvent is present in a concentration in a range of from about 80% v/v to about 85% v/v.

In some embodiments, the aqueous solvent is present in the mother liquid in a concentration less than or equal to about 90% v/v, about 85% v/v, or about 80% v/v. In some embodiments, the aqueous solvent is present in the mother liquid in a concentration in a range of from about 80% v/v to about 85% v/v.

In some embodiments, the halogenation agent is a chlorination agent or a bromination agent. In some embodiments, the halogenation agent is a gaseous halogenation agent. In some embodiments, the halogenation agent is a gaseous chlorination agent or a gaseous bromination agent. In some embodiments, the halogenation agent is selected from the group consisting of gaseous Cl₂, gaseous Br₂, liquid Br₂, and combinations thereof. In some embodiments the gaseous halogenation agent is bubbled into the mixture. In some embodiments, the gaseous halogenation agent is bubbled into the mixture over a period of about 1, 2, 3, 4, 5, or 6 hours. In some embodiments the gaseous halogenation agent is bubbled into the mixture over a period of about 1-6, 2-5, or 2-4 hours. In some embodiments, the halogenation agent is selected from the group consisting of gaseous Cl₂, gaseous Br₂, or a combination thereof and is bubbled into the mixture. In some embodiments the gaseous halogenation agent is bubbled into the mixture over a period of about 2 hours.

In some embodiments, when the halogenation agent is a liquid halogenation agent, the liquid halogenation agent is added to the mixture through dropwise addition.

In some embodiments, the halogenation agent does not comprise hydrochloric acid. In some embodiments, the halogenation agent does not comprise concentrated hydrochloric acid.

In some embodiments, the method step of introducing a halogenation agent to the mixture comprises introducing the halogenation agent to the mixture at a temperature in a range of from about 0° C. to about 35° C.

In some embodiments, the method step of removing the halogenation agent from the mixture comprises bubbling an inert gas through the mixture. In some embodiments, the method step of removing the halogenation agent from the mixture comprises bubbling N2 through the mixture.

In some embodiments, the method produces a solid product, and the method further comprises a method step of filtering and/or washing the solid product. In some embodiments, the method step of filtering and/or washing the solid product comprises washing the solid product with a solution comprising acetic acid and/or water.

In some embodiments, the method further comprises a method step of recovering and/or recycling the aqueous solvent. In some embodiments, the method step of recovering and/or recycling the aqueous solvent recovers and/or recycles at least about 50% v/v, at least about 55% v/v, at least about 60% v/v, at least about 65% v/v, at least about 70% v/v, at least about 75% v/v, at least about 80% v/v, at least about 85% v/v, at least about 90% v/v, at least about 95% v/v, or about 100% v/v of the aqueous solvent.

In some embodiments, the method further comprises a method of step of recovering and/or recycling a mother liquid. In some embodiments, the method step of recovering and/or recycling the mother liquid recovers and/or recycles at least about 50% v/v, at least about 55% v/v, at least about 60% v/v, at least about 65% v/v, at least about 70% v/v, at least about 75% v/v, at least about 80% v/v, at least about 85% v/v, at least about 90% v/v, at least about 95% v/v, or about 100% v/v of the mother liquid.

In some embodiments, the method comprises forming a mixture comprising a compound of Formula I and an optionally recycled aqueous solvent, wherein the aqueous solvent is present in a concentration less than or equal to about 90% v/v, introducing a gaseous halogenation agent to the mixture, reacting the mixture at a temperature in a range of from about 20 to about 35° C., cooling the mixture to a temperature in a range of from about 0° C. to about 5° C., filtering and washing the mixture, and recovering a compound of Formula II in the form of a wet cake. After the filtering and washing step, a minor portion of the mother liquid is optionally subjected to a stripping step to recover acetic acid. The recovered acetic acid is optionally recycled in further reactions. Also after the filtering and washing step, a major portion of the mother liquid is optionally recycled in further reactions.

In some embodiments, the method comprises forming a mixture comprising a compound of Formula I and an optionally recycled aqueous solvent, wherein the aqueous solvent is present in a concentration less than or equal to about 90% v/v, introducing a gaseous halogenation agent to the mixture, reacting the mixture at a temperature in a range of from about 20 to about 35° C., removing the gaseous halogenation agent from the mixture by bubbling N2 through the mixture, cooling the mixture to a temperature in a range of from about 0° C. to about 5° C., filtering and washing the mixture, and recovering a compound of Formula II in the form of a wet cake. After the filtering and washing step, the cake is optionally subjected to a slurry wash with water to yield a first portion wet cake. Also after the filtering and washing step, the mother liquid is optionally subjected to a stripping step at −20 to 60° C. to recover acetic acid and subsequent cooling and filtering and washing steps to further recover acetic acid and yield a second portion wet cake. The recovered acetic acid is optionally recycled in further reactions. The first portion wet cake and the second portion wet cake are optionally combined.

### Examples

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Comparative Example 1. Chlorination with Hydrochloric Acid

120 g dry 2-((ethoxycarbonyl)amino)-3-methylbenzoic acid and 420 g acetic acid are added to a 1 L flask equipped with condenser, thermometer, and dropping funnel. The mixture is stirred and heated to 35-40° C. until all the solid is dissolved. The mixture is then cooled to about 30° C. Next, 124 g 37% hydrochloric acid is added to the mixture and 77 g 30% hydrogen peroxide solution is fed in a period of 3 hours. After addition, the mixture is stirred for another 2 hours until the reaction is completed. 1000 g water is added dropwise into the mixture in a period of 2 hours at 30-35° C., and the product appears during the water addition as a white crystalline. The mixture is cooled to 20-25° C. and the product is given by filter and subsequent water wash. About 124 g 5-chloro-2-((ethoxycarbonyl)amino)-3-methylbenzoic acid product is obtained with about 90% yield.

### Example 1. Chlorination with Chlorine Gas

120 g 2-((ethoxycarbonyl)amino)-3-methylbenzoic acid and 465 g of about 83% acetic acid are added to a 1 L flask equipped with condenser, thermometer, and gas input pipe. Chorine gas is grandually bubbled to the mixture at 0-35° C. at a period of 2 hours and the solid disappears during the gas bubbling process as temperature rises. At the end of stage of gas bubbling, some product appears as white crystalline. After the reaction is finished, the reaction mixture is bubbled with nitrogen to remove excess chlorine. The reaction mixture is cooled to 0-5° C. and stirred for an additional 2 hours. The first portion product is given by filtering and a subsequent 80% acetic acid wash and water wash. The filtrate is concentrated to about 20% volume under reduced pressure at 40-50° C. The residual solution is cooled to 0-5° C. again, and then the second portion product is given by filtering and a subsequent 80% acetic acid wash and water wash. The combined yield of 5-chloro-2-((ethoxycarbonyl)amino)-3-methylbenzoic acid product is about 92%.

### Example 2. Chlorination with Chlorine Gas and Recycling

120 g 2-((ethoxycarbonyl)amino)-3-methylbenzoic acid and 465 g of about 83% acetic acid are added at room temperature to a 1 L flask equipped with condenser, thermometer, and gas input pipe. Chorine gas is bubbled to the mixture at 0-35° C. at a period of 2 hours and the solid disappears during the gas bubbling process as temperature rises. At the end of stage of gas bubbling, some product appears as white crystalline. After the reaction is finished, the reaction mixture is bubbled with nitrogen to remove excess chlorine. The reaction mixture is cooled to 0-5° C. and stirred for an additional 2 hours. The first portion product is given by filtering and a subsequent 80% acetic acid wash and water wash. The yield of 5-chloro-2-((ethoxycarbonyl)amino)-3-methylbenzoic acid product in the first batch product is about 80%.

The filtrate is worked as the solvent of the next batch, and after the same reaction at the same scale and recrystallization process above, more product is given in the second batch. The yield of 5-chloro-2-((ethoxycarbonyl)amino)-3-methylbenzoic acid product in the second batch product is about 95%.

The filtrate of the second batch also can be recycled in third, fourth, and fifth batches. The average yield of the first through fifth batches is about 90-91%.

The methods of the present disclosure reduce the volume of wastewater by about 80%. The methods of the present disclosure also decrease usage of acetic acid by about 80-85%. Therefore, the present methods are particularly beneficial for reducing cost and waste.

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “alkyl”, used either alone or in compound words such as “alkylthio” or “haloalkyl” includes straight-chain or branched alkyl, such as methyl, ethyl, npropyl, ipropyl, or the different butyl, pentyl or hexyl isomers.

The term “alkenyl” can include straight-chain or branched alkenes such as 1propenyl, 2propenyl, and the different butenyl, pentenyl and hexenyl isomers. “Alkenyl” also includes polyenes such as 1,2propadienyl and 2,4hexadienyl.

The term “halogen”, either alone or in compound words such as “haloalkyl”, includes fluorine, chlorine, bromine or iodine.

The total number of carbon atoms in a substituent group is indicated by the “Cᵢ-Cⱼ” prefix where i and j are numbers from 1 to 8. For example, C₁-C₃ alkylsulfonyl designates methylsulfonyl through propylsulfonyl.

When a group contains a substituent which can be hydrogen, for example R⁴, then, when this substituent is taken as hydrogen, it is recognized that this is equivalent to said group being unsubstituted.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

1. A method of preparing a compound of Formula (II) 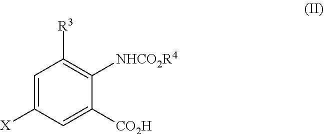
wherein R³ is CH₃ or Cl;
R⁴ is C₁-C₆ alkyl or C₃-C₆ alkenyl, each optionally substituted with up to 3 halogen and up to 1 phenyl; and
X is Cl or Br, the method comprising:
(I) forming a mixture comprising:
A) a compound of Formula (I) 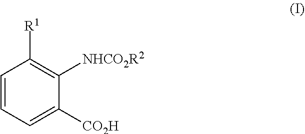
wherein R¹ is CH₃ or Cl; and
R² is C₁-C₆ alkyl or C₃-C₆ alkenyl, each optionally substituted with up to 3 halogen and up to 1 phenyl; and
B) an aqueous solvent, wherein the aqueous solvent is present in a concentration less than or equal to about 90% v/v;
(II) introducing a halogenation agent to the mixture;
(III) optionally removing the halogenation agent from the mixture;
(IV) optionally introducing a strong base to the mixture; and
(V) optionally cooling the mixture to a temperature in a range of from about 0° C. to about 5° C.

2. The method of claim 1, wherein R3 and X are each Cl.

3. The method of claim 1, wherein R³ is CH₃ and X is Cl.

4. The method of claim 1, wherein R⁴ is C₁-C₂ alkyl.

5. The method of claim 1, wherein R¹ is CH₃.

6. The method of claim 1, wherein R² is C₁-C₂ alkyl.

7. The method of claim 1, wherein R³ is CH₃, X is Cl, and R⁴ is ethyl.

8. The method of claim 1, wherein the aqueous solvent is present in a concentration less than or equal to about 85% v/v.

9. The method of claim 1, wherein the aqueous solvent is an aqueous solution of acetic acid.

10. The method of claim 1, wherein the aqueous solvent does not comprise hydrogen peroxide.

11. The method of claim 1, wherein the aqueous solvent comprises a recovered and/or recycled aqueous solvent.

12. The method of claim 1, wherein the halogenation agent is a chlorination agent or a bromination agent.

13. The method of claim 1, wherein the halogenation agent is selected from the group consisting of Cl₂, Br₂, and combinations thereof.

14. The method of claim 1, wherein the method step of introducing a halogenation agent to the mixture comprises introducing the halogenation agent to the mixture at a temperature in a range of from about 0° C. to about 35° C.

15. The method of claim 1, wherein the method step of removing the halogenation agent from the mixture comprises bubbling N2 through the mixture.

16. The method of claim 1, wherein the strong base is selected from sodium hydroxide, potassium hydroxide, sodium carbonate, potassium carbonate, sodium bicarbonate, potassium bicarbonate, and combinations thereof.

17. The method of claim 1, wherein the method produces a solid product, and wherein the method further comprises a method step of filtering and/or washing the solid product.

18. The method of claim 1, wherein the method further comprises a method step of recovering and/or recycling the aqueous solvent.

19. The method of claim 17, wherein the method step of recovering and/or recycling the aqueous solvent recovers and/or recycles at least 50% v/v of the aqueous solvent.

20. The method of claim 1, wherein the method further comprises a method of step of recovering and/or recycling a mother liquid.

21. (canceled)
